(19) United States
(12) Patent Application Publication
(10) Pub. No.: US 2020/0253888 A1
(43) Pub. Date: Aug. 13, 2020

(54) USE OF BIOMARKERS ASSOCIATED WITH LINE-1

(71) Applicant: THE ARIZONA BOARD OF REGENTS ON BEHALF OF THE UNIVERSITY OF ARIZONA, Tucson, AZ (US)

(72) Inventors: Kenneth RAMOS, Tucson, AZ (US); Elsa M. REYES-REYES, Tucson, AZ (US); Pasano BOJANG, Tucson, AZ (US); Patrick Silva, Tucson, AZ (US)

(21) Appl. No.: 16/088,866

(22) PCT Filed: Mar. 28, 2017
(86) PCT No.: PCT/US17/24633

§ 371 Date: Sep. 27, 2018

Related U.S. Application Data
(60) Provisional application No. 62/314,335, filed on Mar. 28, 2016.

(51) Int. Cl. A61K 31/015; C07K 16/18; G01N 33/66; A61P 35/00

(52) U.S. Cl. CPC A61K 31/015; C07K 16/18; G01N 2333/9128; A61P 35/00; G01N 33/66

(57) ABSTRACT

The present invention provides a method for treating a subject having a clinical condition associated with increased level of L1 expression. The method generally includes determining the expression level of L1 in a subject and administering a TGF-β1 inhibitor to a subject having an increased level of L1 expression. The clinical conditions that can be treated using the method of the invention include, but are not limited to, cancer, chronic obstructive pulmonary disease (COPD), atherosclerosis and pulmonary vascular disease as well as other fibrotic and inflammatory diseases.

### CROSS-REFERENCE TO RELATED APPLICATIONS

This application claims the priority benefit of U.S. Provisional Application No. 62/314,335, filed Mar. 28, 2016, which is incorporated herein by reference in its entirety.

### STATEMENT REGARDING FEDERALLY FUNDED RESEARCH

This invention was made with government support under grant number R01 ES017274 awarded by the National Institutes of Health. The government has certain rights in the invention.

### FIELD OF THE INVENTION

The present invention relates to a method for treating a clinical condition associated with aberrant L1 expression by administering a therapeutically effective amount of TGF-β1 inhibitor to a subject suffering from a clinical condition such as cancer, chronic obstructive pulmonary disease (COPD), atherosclerosis and pulmonary vascular disease. The method includes determining the expression level of L1 in a subject and administering a TGF-β1 inhibitor to a subject having increased level of L1 expression.

### BACKGROUND OF THE INVENTION

Long interspersed nuclear element-1 (L1 or LINE-1) is a jumping gene that mobilizes throughout the genome via a ‘copy-and-paste’ retrotransposition mechanism. L1 encodes two proteins essential for its retrosposition activity, ORF1p and ORF2p. L1 activity is silenced in somatic tissues, and its reactivation can be detected in various clinical conditions including, but not limited to, tumors, chronic obstructive pulmonary disease (“COPD”) and fibrotic and inflammatory diseases of the digestive tract, the genitourinary tract, the lung, the kidney, the liver. Exemplary fibrotic and inflammatory clinical conditions include, but are not limited to, Barrett's Esophagus, colitis, asbestosis, asthma, bronchopulmonary dysplasia, emphysema, bronchitis (acute and chronic), primary ciliary dyskinesia, pulmonary fibrosis, silicosis, sarcoidosis, etc.

It is believed that increased expression of L1 promotes pathogenesis by damaging the host DNA via mutation insertions and altering target gene expression and chromosomal rearrangements. Currently, the cellular signal mechanisms promoting the increased L1 expression that leads to disease have not been fully elucidated. Notwithstanding the lack of understanding of mechanism for increased L1 expression, COPD is believed to progress at least in part due to increased L1 expression. Furthermore, many inflammatory diseases are also believed to be caused at least in part by increased L1 expression. In fact, many diseases that share an inflammatory etiology, fibrotic origin, or deficits in cellular adhesion, adhesion diseases and cellular metabolism diseases are believed to be due to increased L1 expression.

Since understanding the cellular level mechanisms leading to increased L1 expression can lead to new therapeutic targets in treating a various clinical conditions associated with increased and aberrant L1 expression, there is a need to determine mechanisms for increased L1 expression.

### SUMMARY OF THE INVENTION

Some aspects of the present invention are based on the discovery by the present inventors of the cellular signal mechanisms leading to increased L1 expression. For example, in development of cancer, the present inventors have discovered that reactivation, and therefore increased L1 expression, is induced by exposure to carcinogens such as benzo(a)pyrene (B[a]P). Experiments have shown that in several human cells, exposure to B[a]P resulted in exogenous expression of L1 , which reprograms the genome of HepG2 cells, leading to the expression of epithelial-to-mesenchymal transition (EMT) markers by L1 retrotransposition dependent and independent functions. In particular, the experiments have shown that L1 expression in HepG2 cells is induced by transforming growth factor-β1 (TGF-β1).

B[a]P challenge experiments showed increased levels of TGF-β1 mRNA via through its receptor—the aryl hydrocarbon receptor (AhR). B[a]P also induced an increase the smad-2 phosphorylation levels and decrease the expression of E-Cadherin and N-Cadherin in HepG2 cells, indicating AhR pathway is able to activate TGF-β1 signal pathway. Using siRNA approaches, the present inventors have found B[a]P-induced L1 expression requires transforming growth factor-β1 receptor 1 (TGFBR1/ALK5) and Smad 2/3 proteins. Moreover, it was found that the expression of L1 proteins overlap with the activation of TGF-β1 signaling pathway in hepatomacellular carcinoma tumors. These results show that AhR activation by B[a]P leads to L1 expression via TGF-β1 signaling pathways acting through Smad-mediated mechanisms. These results also indicate the crosstalk of two important signaling pathways (Ahr and TGF-β1) in liver carcinogenesis following exposure to the carcinogen B[a]P. Similar relationships have been observed in bronchial epithelial cells.

Based at least in part on these findings, some aspects of the invention utilize a TGF-β1 inhibitor to treat a clinical condition associated with increased L1 expression. Any known TGF-β1 inhibitors either alone or in combination can be used to treat a clinical condition associated with aberrant (e.g., increased) L1 expression. Typically, aberrant L1 expression refers to overexpression or increased expression of L1 . Exemplary TGF-β1 inhibitors that are suitable for treating a clinical condition associated with aberrant L1 expression include, but are not limited to, PIRFENIDONE®, GALUNISERTIB®, FRESOLUMIMAB®,TRABEDERSE®, and DISITERTIDE®.

### BRIEF DESCRIPTION OF THE DRAWINGS

FIG. 1 is experimental results showing TGF-β1 induces the L1 mRNA expression in HepG2 cells. HepG2 were stimulated for different times by adding serum-free media with or without (untreated) 10 ng/ml TGF-β1. Total RNA was isolated, and 1 μg of RNA was subjected to cDNA synthesis. Samples were analyzed via RT-PCR using specific primers for human L1 (ORF-1 and ORF-2) and GAPDH. Expression relative to untreated cells.

FIG. 2 is experimental results showing B[a]P induces the of L1 expression in HepG2 cells. HepG2 were stimulated by replacing cell media for supplemented fresh media containing: A) different concentrations of B[a]P or DMSO (0.5%) for 24 h, or B) 1.5 μM B[a]P or DMSO (0.5%) for different periods of times. Total RNA was isolated, and 1 μg of RNA was subjected to cDNA synthesis. Samples were analyzed via RT-PCR using specific primers for human L1 (ORF1 and ORF2), and GAPDH. C) Some cells were lysed and whole cell lysates were analyzed by immunoblotting for L1 proteins (ORF1p and ORF2p), AhR and GAPDH (loading control).

FIG. 3 is experimental results showing B[a]P induces the TG-β1 mRNA expression through of its receptor AhR in HepG2 cells. A) Cell were treated with 1.5 μM B[a]P or an equivalent DMSO concentration (0.5%) for different periods of times. B) Cells were transfected without siRNA (mock), with AhR target-specific siRNAs, or with a control siRNA (scramble), then treated with 1.5 μM B[a]P or DMSO for 48 h. Total RNA was isolated, and 1 μg of RNA was subjected to cDNA synthesis. Samples were analyzed via RT-PCR using human specific primers for TGF-μ1 and GAPDH. C) Some transfected cells were lysed and the whole cell lysates was analyzed by immunoblotting for AhR or GAPDH antibodies (loading control) to confirm target knockdown.

FIG. 4 is experimental results showing B[a]P induces the activation of the TGF-β1 signaling pathway in HepG2 cells. Cells were stimulated for different periods of times by replacing cell media for supplemented fresh media containing 1.5 μM B[a]P or an equivalent DMSO concentration (0.5%). Whole cell lysates were analyzed by immunoblotting for phospho-Smad2, total Smad2, E-Cadherin, N-Cadherin, and GAPDH (loading control).

FIG. 5 is experimental results showing B[a]P-mediated L1 mRNA expression requires TGFR1, Smad2 and smad3 in HepG2 cells. Cells were transfected without siRNA (mock), with target-specific siRNAs for Smad2, Smad3, TGFBR1, or with a control siRNA (scramble), then treated with 1.5 μM B[a]P or DMSO for 48 h. Total RNA was isolated, and 1 μg of RNA was subjected to cDNA synthesis. Samples were analyzed via RT-PCR using human specific primers for L1 (ORF1 and ORF2) and GAPDH. C) Some transfected cells were lysed and the whole cell lysates was analyzed by immunoblotting for TGFBR1, Smad2/3 or GAPDH antibodies (loading control) to confirm target knockdown.

FIG. 6 is experimental results showing expression of L1 proteins overlap with the activation of TGF-β1 signaling pathway in liver tumors. Whole protein extract from human liver hepatocellular carcinoma stages I, II, III, IV, and normal tissue were analyzed as follow: A) Immunoblotting for L1 proteins (ORF1p and ORF2p), vimentin, E-cadherin, Vimentin, snail and GAPDH (as loading control). B) Immunoblotting for phospho-Smad2 and total Smad2/3.

FIG. 7 is a schematic illustration of a possible mechanism for AhR and TGF-β1 crosstalk on the regulation of L1 expression induced by the carcinogen B[a]P in HepG2 cells. AhR activation by B[a]P leads to L1 expression via TGF-β1 signaling pathways acting through Smad-mediated mechanisms. These findings define the crosstalk of two important signaling pathways (Ahr/TGF-β1) in carcinogenesis following exposure to the carcinogen B[a]P.

FIG. 8 is a graph showing results of L1 ORF1p expression in TESAOD cohorts.

FIG. 9 shows three separate experimental results all showing TGF-β1 induces the expression of L1 and the presence of Juglone, which is a naturally occurring inhibitor of TGF-β1, inhibits TGF-β1-mediated L1 expression. In the experiments, non-malignant BEAS-2B lung cells were untreated or treated with 10 ng/ml TGF-β1 and co-treated with 1 μM juglone or ethanol and whole lyses were analyzed by immunoblotting for L1 proteins (ORF1p and ORF2p), vimentin, DNMT1 and GAPDH (loading control).

### DETAILED DESCRIPTION OF THE INVENTION

Long interspersed nuclear element-1 (LINE-1 or L1) is a repetitive DNA sequence abundant in the mammalian genome that mobilizes by retrotransposition via a “copy and paste” mechanism. A functional L1 element in humans is 6 kb in length and consists of a 5′ untranslated region (UTR) with promoter activity, two open reading frames, and a terminal 3′ UTR (1, 2). L1 encodes two proteins; ORF1p is a 40 kDa protein with nucleic acid binding activity, while ORF2p is a 150 kDa protein with endonuclease and reverse transcriptase activities (3). A complete L1 retrotransposition cycle consists of transcription of L1 RNA, export into the cytoplasm, translation of ORF1 and ORF2, association of L1 RNA with ORF1 and ORF2 proteins to form ribonucleoprotein (RNP) particles, return to the nucleus, reverse transcription, and integration at a new genomic location (4).

L1 retrotransposition cycle is tightly regulated in somatic tissues (5) by genetic and epigenetic mechanisms (6-8). However, aberrant (i.e., increased) expression of ORF1p (9) and new somatic L1 insertion in the genome have been detected in several epithelial cancers. L1 reactivation exerts retrotransposition-dependent and retrotransposition-independent functions that is believed to promote neoplastic transformation and cancer progression (10). L1 retrotranspostion function has mutagenic potential. By inserting in the genome, it is believed that L1 promotes carcinogenesis by inducing aberrant splicing, exon skipping, and genome rearrangements that change gene expression or lead to genomic instability (11-13). It is also believed that L1 retrotransposition-independent function modulates cancer progression by impacting gene expression.

The present inventors have shown that ectopic expression of a mutant L1 incapable of retrotransposition modulates the expression of a large number of genetic targets involved in cancer progression, including adhesion, inflammation and cellular metabolism pathways (14, 15). Expression of mutant L1 has also found to disrupt epithelial cell differentiation and induces epithelial-to-mesenchymal transition (EMT), which is a process that facilitates metastasis by promoting cell motility and conferring malignant cells the ability to invade.

One master promoter of the EMT (16) is the transforming growth factor-β1 (TGF-β1), which is also one of the most potent immunosuppressive and pro-inflammatory cytokines (17). TGF-β1 plays a dual role in the process of carcinogenesis. On one hand, it can inhibit proliferation of cancer cells; on the other, it can activate migration and invasion of cancer cells due the induction of the EMT (16) or through a direct stimulation of neoangiogenesis in tumor tissue (18). TGF-β1 signaling pathway is mediated via type I and type II transmembrane receptors with serine/threonine kinase activity. TGF-β1 signal is transduced from the cell membrane to the nucleus canonically through the complex of SMAD proteins or non-canonically through other signaling pathways, e.g., Jun N-terminal kinase (JNK), p38 and extracellular signal-regulated kinases/mitogen-activated protein kinases (ERK/MAPK) (19).

Using in silico analysis of the regulatory genetic network for L1 (15), and in vitro validation in HepG2 cells (14), the present inventors have identified that some of the genetic targets of L1 are also regulated by TGF-β1 signaling pathway (e.g., CCL2, ICAM CXCL1). These data show TGF-β1 signaling pathway is involved in some of the carcinogenesis functions of L1.

Based on these finding, the present inventors have discovered that modulation of TGF-β1 signaling pathway can be used to treat clinical conditions associated with aberrant L1 expression. Some of the clinical conditions that are associated with aberrant L1 expression include, but are not limited to, tumors; chronic obstructive pulmonary disease (“COPD”); cell or tissue damage due to environment/toxin exposure; fibrotic and inflammatory diseases of the digestive tract, the genitourinary tract, the lung, the kidney, the liver, including Barrett's Esophagus, colitis, asbestosis, asthma, bronchopulmonary dysplasia, emphysema, bronchitis (acute and chronic), primary ciliary dyskinesia, pulmonary fibrosis, silicosis, sarcoidosis, etc. Exemplary tumors or cancer that can be treated by the method of the invention include, but are not limited to, lung cancer, bladder cancer, esophageal cancer, gastric cancer, hepatic cancer, breast cancer, prostate cancer, colorectal cancer, testicular cancer, renal cancer, chronic lymphocytic leukemia, chronic myeloid leukemia, ovarian carcinomas, and human papilloma virus related cancers.

In some embodiments, the method of the invention includes determining whether a subject has aberrant L1 expression. This determination can be achieved using a variety of methods including, but not limited to, determining the expression level of L1 , determining the level of L1 mRNA present in a sample, determining the level of proteins encoded by L1 (e.g., ORF1p and/or ORF2p) present in a sample, etc. One particular method of determining the level of ORF1p is disclosed in PCT Patent Application Publication No. WO 2014/004945, which is incorporated herein by reference in its entirety. Another method of determining the level of L1 expression is quantitative real time polymerase chain reaction (“qRT-PCR”) as disclosed in the Examples section. Such qRT-PCR can be achieved using the primers disclosed herein. Other specific examples of methods for determining L1 expression include, but are not limited to, immunoassay immunoblot, (e.g., ELISA using an antibody of ORF1p and/or ORF2p), MALDI mass spectrometry measuring ORF-1 protein and/or ORF-2 protein; antibody or aptamer array measuring ORF-1 protein and/or ORF-2; gene expression microarrays measuring LINE-1 mRNA; RNAseq measuring LINE-1 mRNA; barcode counter gene expression technology. In general, any method that can detect ORF-1 and/or ORF-2 as well as mRNA of L1 as well as the number of L1 present can be used in methods of the invention.

The L1 expression level of a subject can be determined using any fluid or cell sample obtained from the subject, including, but not limited to, blood, serum, plasma, saliva, mucus, pleural effusion, solid tissue biopsy, liquid biopsy, bronchial brush, bronchial wash, and bladder cytology . In addition, the L1 expression level can be obtained from cell sample obtained from the subject, e.g., biopsy cells, etc.

In some embodiments, the expression level of L1 is compared to a control L1 expression level. As used herein, the term “control L1 expression level” can be expression level of L1 in subjects (or “normal groups”) that do not have any clinical conditions described herein. Alternatively, the “control L1 expression level” can be the minimum L1 expression level from subjects in the early stages of the clinical conditions described herein. For example, in blood serum analysis, the “normal”, i.e., subjects who do not have a clinical condition associated with increased L1 expression is about 5 ng/μL or less, often no more than 10 ng/μL or less. The term “about” when referring to a numerical value refers to ±20%, typically ±10%, often ±5%, and most often ±2% of the numerical value. Alternatively, a level of L1 expression greater than about 10 ng/μL or higher in blood serum, and often about 15 ng/μL or higher can be considered to be having an increased L1 expression. It should be appreciated that these control values can change depending on the sensitivity of the analytical methodology used and as more data is gathered. As with any statistical data, as the number of data points increases, the accuracy of the control value also increases.

The term “control L1 expression level” can also include a normal or negative control and/or a disease or positive control, against which a test level of L1 expression can be compared. Therefore, it can be determined, based on the control level of L1 expression, whether a sample to be evaluated for treatment with TGF-β1 has a measurable difference or substantially no difference in L1 expression, as compared to the control or baseline level. In one embodiment, the control or baseline L1 expression level is indicative of the level of L1 expression as expected in the normal (e.g., healthy or negative control) subject. Therefore, the term “negative control” used in reference to a baseline level of L1 expression typically refers to a baseline level of L1 expression from a population of individuals which is believed to be normal (i.e., subjects not having or developing a clinical condition associated with increased L1 expression). In some embodiments of the invention, it may also be useful to compare the L1 expression in a test sample to a baseline that has previously been established from a patient or population of patients with clinical conditions associated with increased L1 expression. Such a baseline level, also referred to herein as a “positive control”, refers to a level of L1 expression established in subjects suffering from a clinical condition associated with increased L1 expression.

In one embodiment, when the goal is to monitor the progression or regression of a disease associated with increase L1 expression in a subject, for example, to monitor the efficacy of treatment of the disease or to determine whether a subject that appears to be predisposed to the disease begins to develop the disease, one baseline control can include the measurements of L1 expression in a sample from the subject that was taken from a prior test in the same subject. In this embodiment, a new sample is evaluated periodically (e.g., at annual or more regular physicals), and any changes in L1 expression in the subject as compared to the prior measurement and most typically, also with reference to the above-described normal and/or positive controls, are monitored. Monitoring of a subject's L1 expression profile can be used by the clinician to prescribe or modify treatment for the patient based on whether any differences in L1 expression in the subject is indicated.

Still in another embodiment, when the goal is to determine whether a subject has been exposed to a particularly harmful agent (e.g., B[a]P, biochemical agents, environmental contaminants, radiation, or infection), one can compare the pre-exposure level of L1 expression in the subject to a post-exposure level of L1 expression. For example, one can measure L1 expression of a subject prior to entering a potentially harmful site and measure the subject's L1 expression level after having entered the site. If the L1 expression level is increased (e.g., by at least about 5%, typically by at least about 10%, often by at least about 25%, more often by at least about 50%, and most often by at least about 100%), then it is likely that the subject has been exposed to a potentially dangerous agent. In some embodiments, such an increase in L1 expression can be an indication of a likelihood of or increased probability of pathogenic structural genome changes and/or gene expression changes. If the L1 expression level is substantially the same (i.e., within about ±20%, typically within about ±10%, and often within about ±5%), then it is likely that the subject has not been exposed to a potentially dangerous agent at harmful levels or that exposure has a low potential for or no significant likelihood of structural genome changes and/or gene expression changes. One can also monitor the subject's L1 expression level yearly or periodically to determine whether the subject has been exposed to any dangerous agents. Such a test can be used, for example, to monitor whether the subject is complying with the safety measurements that are promulgated for a given occupation.

In some aspects of the invention, the level of L1 expression is used to determine whether exposure to a harmful agent has any deleterious effect on a subject's health. This can be achieved, for example, by comparing the L1 expression level of a subject who has been exposed to a harmful agent with the control L1 expression level. The control L1 expression level can be the subject's own L1 expression level prior to or immediately after exposure to the harmful agent. As used herein, the term “immediately” in connection with exposure to a harmful agent means within a week, typically within three days or less, often within a day, and often within a few hours (e.g., 10 hours or less, typically 5 hours or less, and often 3 hours or less) after exposure to a harmful agent. The L1 expression level can be monitored regularly, e.g., weekly, monthly, or yearly, to determine whether there is any immediate or long term deleterious affect on the subject's health due to exposure to the harmful agent. Exemplary harmful agents include, but not limited to, radiation (e.g., due to nuclear waste exposure or nuclear reactor accident), pathogens (e.g., bacteria, fungi, etc.), toxins (e.g., toxic chemical compounds such as nerve agents, biochemical weapons, carcinogens, etc.) as well as exposure to unknown or unidentified substances. The control L1 expression level can also be the previously measured L1 expression level of the subject. For example, if the subject is being monitored weekly, monthly, yearly or at some regular intervals, one can compare the L1 expression level change in the subject from the previous L1 expression level measurement. In this manner, one can determine whether exposure to the harmful agent is having any deleterious affect on the subject's health. The control L1 expression level can also be either a positive or negative control L1 expression level as defined herein.

Another aspect of the invention provides a method for determining a treatment protocol for a patient suffering from various clinical conditions disclosed herein. Such a method includes measuring the L1 expression level in the patient. If the patient shows increased L1 expression, then the treatment protocol includes administering TGF-β1 inhibitor. For example, a breast cancer patient can be treated with a variety of chemotherapeutic agents and/or radiotherapy. However, by measure the patient's L1 expression level, one can readily determine whether treatment with a TGF-β1 inhibitor will likely be successful or whether the treatment should include a TGF-β1 inhibitor.

In one particular embodiment, the control or baseline levels of L1 expression are obtained from “matched individuals”. According to the present invention, the phrase “matched individuals” refers to a matching of the control individuals on the basis of one or more characteristics, such as gender, age, race, or any relevant biological or sociological factor that may affect the baseline of the control individuals and the subject (e.g., preexisting conditions, consumption of particular substances, levels of other biological or physiological factors). The number of matched individuals from whom control samples must be obtained to establish a suitable control level (e.g., a population) can be determined by those of skill in the art, but should be statistically appropriate to establish a suitable baseline for comparison with the subject to be evaluated (i.e., the test patient). The values obtained from the control samples are statistically processed using any suitable method of statistical analysis to establish a suitable baseline level using methods standard in the art for establishing such values. It will be appreciated by those of skill in the art that a baseline need not be established for each assay as the assay is performed but rather, a baseline can be established by referring to a form of stored information regarding a previously determined control level of L1 expression. Such a form of stored information can include, for example, but is not limited to, a reference chart, listing or electronic file of population or individual data regarding “normal” (negative control) or positive L1 expression; a medical chart for the subject recording data from previous evaluations; or any other source of data regarding control L1 expression that is useful for the subject to be diagnosed or evaluated.

The method of the invention also includes treating the subject having an increased L1 expression with a TGF-β1 inhibitor. “Treating” or “treatment” of a disease includes: (1) preventing the disease, i.e., causing the clinical symptoms of the disease not to develop in a subject that may be exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms. Exemplary TGF-β1 inhibitors that are useful in method of the invention include, but are not limited to, PIRFENIDONE®, GALUNISERTIB®, FRESOLUMIMAB®,TRABEDERSE®, JUGLONE® and DISITERTIDE®.

In one specific example, as discussed herein, the present inventors have shown that L1 reactivation can be induced by B[a]P, a polycyclic aromatic hydrocarbon. B(a)P is a mutagen and carcinogen environmental contaminant with diverse toxicological effects (20). The biological effects of B[a]P is mainly mediated through its binding to the aryl hydrocarbon receptor (AhR). The AhR is a ligand-activated transcription factor ubiquitously distributed in the body, and after ligation of dioxins to the AhR, the receptor translocates from the cytosol to the nucleus, heterodimerizes with the AhR nuclear translocator (ARNT), and binds to an enhancer sequence, called a dioxin response element (DRE), of several drug metabolizing enzymes, such as cytochrome P450 1A1 (CYP1A1)(21).

There exists a cell-specific and context-dependent crosstalk between AhR and transforming growth factor-β1 (TGF-β1) signaling pathway, both AhR and TGF-β1 participate in regulation of common cellular processes e.g. cell cycle machinery, apoptosis, cell adhesion and interaction with extracellular matrix (ECM) (22). Several studies make evident that AhR can regulate TGF-β1 signaling, through various mechanisms involving deregulation of TGF-β1 secretion, suppression or increase of TGF-β1 expression or down-regulation of the latency-associated protein (LTBP-1) expression (23-25). TGF-β1 also regulates AhR expression and CYP1A1/1B1 enzyme activity in a cell/tissue specific manner (26-28). Thus, different mechanisms been proposed to explain the function of AhR and TGF-β1 crosstalk in several cells systems e.g. in endothelium (29), regulatory T cells (30), Th17 cells (31) or dendritic cells (32). Therefore, it is believed that cell- and tissue-context specific mechanisms play a key role in the outcome of the crosstalk between the TGF-β1 and AhR signaling.

The principles that regulate AhR and TGF-β1 crosstalk seem to be very complex and are still not fully understood. As discussed in detail in the Examples section, the present inventors have observed that B[a]P induces the expression of L1 in several cell lines (33), and L1 induces EMT and disrupts chemokine expression in hepatic carcinoma cells via retrotransposition-dependent and independent mechanisms (14, 15). Due the biological effects of B[a]P are mainly mediated through binding to the AhR, and TGF-β1 is a master molecule to induce EMT, in this study we investigated whether crosstalk between AhR and TGF-β1 might regulator the expression of L1.

The knowledge and insights of LINE-1 biology and transcriptional control gained over the last 20 years are beginning to spawn assays, decision rules, molecular probes, therapeutic modalities and digital tools that enable the development of many of the diagnostic and therapeutic products that are becoming clinically available for use (e.g., TGF-β1 inhibitors).

The method of the invention is based at least in part by the discovery of the underlying mechanism of aberrant L1 expression to provide drug discovery, biomarker validation, and assay validation methods. The method of the invention also allows discovery of new diagnostic tools and therapeutic modalities. Such a method also allows expanded use of clinical stage and approved drugs (that block the effects of TGF-β1), as well as reformulation of TGF-β1 inhibitors used for other diseases.

In one particular embodiment, the method of the invention is used for the early diagnosis and treatment of cancers, and especially, squamous cell carcinoma of the lung, with classes of drugs that are in clinical trials or approved for use (drugs that block the actions of TGF-β1). In addition, TGF-β and aberrant ORF-1 protein expression have been implicated in driving the pathogenic phenotype in COPD. Accordingly, the method of the invention is also useful in determining treatment protocol for COPD. In some embodiments, TGF-β1 inhibitor is delivered via airway (e.g., as an aerosol or a mist), for at-risk COPD or early diagnosis lung cancer cases.

Another aspect of the invention includes L1 gene expression silencing to treat a clinical condition associated with aberrant L1 expression level. Such a gene expression silencing can be achieved by using siRNA, miRNA/antagimirs, antisense oligonucleotides against L1 , TGF, its receptors, SMAD proteins, etc. small molecules that modulate gene expression by interacting with G-quadraplex and I motif elements of the promoters of these target genes.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

Benzo[a]pyrene (B[a]P) was purchased from Ultra Scientific (Kingstown, RI). Recombinant human TGF-β1 was purchase from R&D Systems (Minneapolis, Minn.). Rabbit anti-LINE-1 (H-110), monoclonal anti-GAPDH, and horseradish peroxidase (HRP) linked anti-mouse IgG antibodies were from Santa Cruz Biotech (Dallas, Tex.). Rabbit anti-AhR (13790), anti-E-cadherin (24E10), anti-N-cadherin (13116), anti-vimentin (D21H3), anti-Smad2 (5339) anti-phospho-Smad2 (3108), anti-Smad2/3 (8685), anti-TGFRB1 (3712), and horseradish peroxidase (HRP) linked anti-rabbit IgG antibodies were from Cell Signaling Technology (Beverly, Mass.). Protein lysates from: normal limits liver tissue (male, case ID. CU0000001489, Cat No. CP565754), staging I hepatocellular liver carcinoma tissue (male, case ID. CU0000012132, Cat No. CP641361), staging II hepatocellular liver carcinoma tissue (male, case ID. CU0000005407, Cat No. CP19427), staging IIIA hepatocellular liver carcinoma tissue (male, case ID. CU000001197, Cat No. CP607175), and staging IV hepatocellular liver carcinoma tissue (male, case ID. CU0000013002, Cat No. CP532216) were purchased from OriGene (Rockville, Md.). DMSO was from American Type Culture Collection (ATCC).

Polyclonal anti-Human ORF1p antibody: A custom made polyclonal antibody was produced by New England Peptide LLC to meet specifications with a sequence of H₂N-MGKKQNRKTGNSKTC-amide corresponding to amino acids 1 to 14 of L1 ORF1p. After immunization of two BALB/c mice with the conjugated peptide, three separate bleeds were harvested from each animal. The bleeds were combined and then taken through an affinity purification process. The primary antibody purified antibody (0.224 mg/mL) was aliquoted into 25 μL volumes and stored at −80° C. The antibody is diluted 1:1000 to be used in the assay. The purified antibody was validated in Western blot against L1 ORF1p expressed from plasmid constructs.

Cell culture and treatments: HepG2 hepatocellular carcinoma cell line was purchased from the American Type Culture Collection (ATCC). Cells were grown in RPMI medium supplemented with 10% fetal bovine serum (FBS, Thermo Fisher Scientific, Grand Island, N.Y.) supplemented with 62.5 μg/mL penicillin and 100 μg/mL streptomycin (Thermo Fisher Scientific) in a humidified incubator at 37° C. with 5% CO2. HepG2 cells were pleated on day before treatment. Cells were between 40-50% confluent at the treatment time. B[a]P was dissolved in DMSO to have 1 mM stock solution, and cells were treated by replacing cell media for complete fresh media containing different concentration of B(a)P. For TGF-β1 treatment, HepG2 cells were treated by washing cells once in serum-free media and adding serum-free media containing 10 ng/ml TGF-β1. For biochemical analyses, cells were lysed in cell lysis buffer [150 mmol/L NaCl, 2 mmol/L EDTA, 50 mmol/L Tris-HCl, 0.25% deoxycholic acid, 1% IGEPAL CA-630 (pH 7.5)] containing protease and phosphatase inhibitor cocktails (EMD Millipore) for 5 minutes at 4° C. and then cleared by centrifugation at 16,000×g for 10 minutes at 4° C. All protein concentrations were determined using the bicinchoninic acid assay (Thermo Fisher Scientific).

Immunoblotting: Total cell lysates were resolved by SDS-Tris PAGE and then electrotransferred onto polyvinylidine fluoride membranes (Thermo Fisher Scientific) in Tris-glycine buffer containing 20% methanol. Proteins were detected by immunoblotting. Where indicated, membranes were stripped of bound antibodies using 62.5 mmol/L Tris-HCl (pH 6.7), 100 mmol/L 2-mercaptoethanol, and 2% SDS for 30 minutes at 60° C. and then reprobed as described in figure legends.

RT-PCR: Total RNA was isolated from cells using the RNeasy Plus Kit (Qiagen) and 2 μg RNA was digested with TurboDNase-I (Thermo Fisher Scientific). A total DNAse digested RNA (1 μg) was employed for cDNA synthesis using high-capacity cDNA reverse transcription Kit (Thermo Fisher Scientific). The resulting cDNAs (50 ng) were used as templates for qRT-PCR to analyze the mRNA expression using Power SYBR® Green PCR Master Mix and primers for L1-ORF-1, L1-ORF-2, TGF-β1 and GAPDH. The specific sequences are indicated in the Table 1. The fold changes were determined by comparing the ACT value of each product normalized to GAPDH as an internal control.

TABLE 1

| Sequence | Description |
|---|---|
| 5′-CCA AGTTGGAAAACACTCTGC-3′ | ORF1-Forward-RT- |
| (SEQ ID NO: 1) | PCR |
| 5′-TGTGGCGTTCTCTGTATTTCC-3′ | ORF1-Reverse-RT- |
| (SEQ ID NO: 2) | PCR |
| 5′-TCGACACATACACTCTCCCAAG-3′ | ORF2-Forward-RT- |
| (SEQ ID NO: 3) | PCR |
| 5′-TGGTCCTGGACTCTTTTTGG-3′ | ORF2-Reverse-RT- |
| (SEQ ID NO: 4) | PCR |
| 5′-GGATACCAACTATTGCTTCAGCTCC-3′ | TGFβ1-Forward-RT- |
| (SEQ ID NO: 5) | PCR |
| 5′-AGGCTCCAAATATAGGGGCAGGGTC-3′ | TGFβ1-Reverse-RT- |
| (SEQ ID NO: 6) | PCR |
| 5′-GATCATCAGCAATGCCTCCT-3′ | GAPDH-Forward-RT- |
| (SEQ ID NO: 7) | PCR |
| 5′-TGTGGTCATGAGTCCTTCCA-3′ | GAPDH-Reverse-RT- |
| (SEQ ID NO: 8) | PCR |
| 5′-CGGAUGAAAUCCUGACGUAtt-3′ | AHR-siRNA-Sense |
| (SEQ ID NO: 9) | |
| 5′-UACGUCAGGAUUUCAUCAGtt-3′ | AHR-siRNA-Anti- |
| (SEQ ID NO: 10) | sense |
| 5′-CAGUUACUGUGGAAGGAAUtt-3′ | TGFBR1-siRNA-Sense |
| (SEQ ID NO 11) | |
| 5′-AUUCCUUCCACAGUAACUGTg-3′ | TGFBR1-siRNA-Anti- |
| (SEQ ID NO: 12) | sense |
| 5′-GCUUCUCUGAACAAACCAGtt-3′ | SMAD2-siRNA-Sense |
| (SEQ ID NO: 13) | |
| 5′-CUGGUUUGUUCAGAGAAGCtg-3′ | SMAD2-siRNA-Anti- |
| (SEQ ID NO: 14) | sense |
| 5′-GGCCCAGUGCAUAUGCAAUtt-3′ | SMAD3-siRNA-Sense |
| (SEQ ID NO: 15) | |
| 5′-AUUGCAUAUGCACUGGGCCtc-3′ | SMAD3-siRNA-Anti- |
| (SEQ ID NO: 16) | sense |

RNA interference: Small interfering RNA (siRNA) duplexes sequences were chemically synthesized and annealed by Thermo Fisher Scientific. The siRNA duplexes for AhR (ID#s1200), Smad2 (ID#115715), Smad3 (ID#107877), and TGFBR1 (ID#s22939) corresponded sequences are indicated in the Table 1. BLAST analysis showed no homology to any sequence in the Human Genome Database, other than the intended target. The scrambled siRNA used were Silencer® Negative Control #1 siRNA (AM4635) and Silencer® Select Negative Control #2 siRNA (4390847). The siRNAs were transfected using Lipofectamine™ RNAiMAX (Thermo Fisher Scientific), according to the manufacturer's directions.

Analysis of L1 in COPD Patients:

L1 levels in 428 COPD patients were measured using the following procedure. L1-peptide dilutions (56, 48, 40, 32, 24, 16, 8, 0 pg/mL) were prepared in carbonate buffer. Briefly, to 40 μL of carbonate buffer was added 10 μL of each standard dilution in columns 1 and 12 of the table below.

| STD-1 | S-1 | S-2 | S-3 | S-4 | S-5 | S-6 | S-7 | S-8 | S-9 | S-10 | STD-12 |
|---|---|---|---|---|---|---|---|---|---|---|---|
| 0 | — | — | — | — | — | — | — | — | — | — | 0 |
| 4 | — | — | — | — | — | — | — | — | — | — | 4 |
| 6 | — | — | — | — | — | — | — | — | — | — | 6 |
| 8 | — | — | — | — | — | — | — | — | — | — | 8 |
| 12 | — | — | — | — | — | — | — | — | — | — | 12 |
| 16 | — | — | — | — | — | — | — | — | — | — | 16 |
| 20 | — | — | — | — | — | — | — | — | — | — | 20 |
| 24 | — | — | — | — | — | — | — | — | — | — | 24 |

S denote sample and can be replaced by sample number.

Following procedure was used in coating and dilution: (1) Solubilize antigen in carbonate buffer (1/200 dilution); (2) Dilute the solubilize antigen in 1/400; (3) Add 40 μl of carbonate buffer to each well of a 96 well plate; (4) Add 10 μl of each sample (1/400) dilution into each well of a 96 well plate (i.e. from column 2-11); (5) Use column 1 and 12 for standard dilutions; and (6) Incubate overnight @4° C. This is assuming that the absorbance read for the diluted sample is in the linear range of standard.

Afterwards, samples were washed 3× with PBS/0.05% Tween-20 using the program: ELISA-6. To each well was added 100 μL of SuperBlock (Pierce 37515) (or 2% BSA). The mixture was incubated at 1 hr @37° C.; 2 hr @RT; or overnight @4° C. The samples were then washed 3× with PBS/0.05% Tween-20 using the program: ELISA-6.

L1 ORF1 antibody was diluted 1/500 in 50/50 blocking/wash buffer and 50 μL of the antibody solution was added to each well of a 96 well plate. The resulting mixture was incubated 1 hr @37° C.; 2 hr @RT; or overnight @4° C. Samples were then washed 3× with PBS/0.05% Tween-20 using the program: ELISA-6.

A dilute Goat a-Rabbit IgG-HRP (Rockland 611-1324) @1:5000 in 50/50 blocking/wash buffer solution was prepared. 50 μL of this dilute secondary antibody-enzyme conjugate solution was added to each well of a 96 well plate and incubate 1 hr @37° C.; 2 hr @RT; or overnight @4° C. The samples were again washed 3× with PBS/0.05% Tween-20 using the program: ELISA-6.

Substrates were freshly mixed in TMB (Pierce 34021), and 50 μL of the mixture was added to each well and incubated 10-15 min at RT. 50 μL of 1N H₂SO₄ solution was added to each well and OD was read at 450 nm in Cytation-3 machine.

### Results

Expression of L1 is induced by B[a]P: Experimental results show that L1 promotes cancer pathogenesis and progression by, at least in part, damaging the host DNA via mutation insertions and altering in target gene expression and chromosomal rearrangement. The results also showed that ectopic expression of a mutant L1 (unable to be inserted in the genome) induced EMT and disrupted chemokine expression in the hepatic carcinoma cell line HepG2 (14). These data support that finding that L1 expression is a factor in promoting malignant transformation and cancer progression. TGF β1 is a master promoter of the EMT (16) and one of the most potent immunosuppressive and pro-inflammatory cytokine (16, 17). Previously, no evidence was found that ectopic L1 expression changes the expression of TGF-β1 and its receptors (TGFRI/II) (14, 15). In order to determine whether TGF-β1 was able to induce L1 mRNA expression, HepG2 cells were treated with 10 ng/ml TGF-β1 in serum-free media for different time periods (4 h-24 h) (FIG. 1). The expression of L1 mRNAs was measure by qRT-PCR. TGF-β1 treatment induced an evident increase in the expression levels of L-1 mRNAs (ORF-1 and ORF-2) after 8 h to 24 h treatment, observing the maxima peak induction at 8 h (FIG. 1). These results show that TGF-β1 signaling pathway is at least one of the cellular mechanisms regulating L1 expression.

AhR activation by B[a]P regulates the expression L1 in HepG2 cells: It is well established that there exists a cell-specific and context-dependent crosstalk between AhR and TGF-β1 signaling pathways to regulate several cellular functions. The present inventors have previously shown that the AhR activation by its ligand B[a]P (a mutagen and carcinogen environmental) reactivates transcriptional expression of L1 in several cell lines (e.g., HeLa, HMEM, vSMC and mK4) (33). To determine whether AhR and TGF-β1 signaling pathways crosstalk to regulate L1 expression, the capacity of B[a]P treatment to induce the expression of L1 in HepG2 cells was analyzed. To this end, these cells were challenged with different concentration of B[a]P (FIG. 2A) for different time intervals as indicated in FIG. 2B. Expression levels of L1 mRNAs and proteins (ORF1p and ORF2p) were analyzed by qRT-PCR and immunoblotting respectively. The maximal induction levels of L1 mRNA (ORF1 and ORF2) by B[a]P was observed at a concentration of 1.5 μM (24 h of treatment) (FIG. 2A) and after 48 h of 1.5 μM B[a]P treatment (FIG. 2B). A strong cell cytotoxicity was observed after this time point. Immunoblotting analysis showed that B[a]P treatment for 8-24 h provoked a decrease in the expression of its receptor AhR (FIG. 2C), a response consistent with ubiquitination and subsequent degradation of AHR following ligand activation (34). The expression of L1 proteins (ORF-1p and ORF-2p) were detected after 48 h of B[a]P treatment, coinciding with the expression restoration of AhR (FIG. 2C). These results indicate that L1 protein expression by B[a]P is a secondary response that might require the activation and expression restoration of AhR. These data are also in agreement with the present inventors previous findings, suggesting that B[a]P carcinogen may be an inducer of the L1 reactivation.

AhR activation by B[a]P increases TGF-β1 mRNA levels and induce activation of TGF-β1 signaling pathway: Given that several reports have implicated the AhR in the regulation of TGF-β signaling, experiments were conducted to determine whether AhR activation by B(a)P challenge regulates the expression of TGF-β1 cell models. HepG2 were treated with 1.5 μM B[a]P for 12 h, 24 h and 48 h, and the expression of TGF-β1 mRNA was analyzed by qRT-PCR. TGF-β1 mRNA levels was increased by B[a]P after 12 h, maximum increase was observed after 48 h of treatment (FIG. 3A). B[a]P-mediated TGF-β1 mRNA expression (FIG. 3A) appeared to have started earlier than or coincided with B[a]P-mediated L1 protein expression (FIG. 2). To further define whether AhR activation was required on the B[a]P-induced TGF-β1 mRNA expression, expression of AhR was knocked down by transfection of its specific siRNA in HepG2 cells. Immunoblot analysis confirmed >90% reduction in levels compared with controls (FIG. 3C). As shown in FIG. 3B, inhibition of AhR expression caused a substantial decrease (p<0.05) in B[a]P-induced TGF-β1 mRNA expression. These results indicate that AhR activation is required for in B[a]P-induced TGF-β1 mRNA expression.

Whether AhR-induced TGF-β1 expression led to the TGF-β1 pathway activation was also examined by analyzing typically downstream of TGF-β1, such as activation of Smad2, expression of EMT markers (N-Cadherin, vimentin) and downregulation expression of the epithelial marker E-cadherin. Immunoblotting showed that B[a]P challenge was able to increase phosphorylation of Smad2 and downregulated the expression of E-Cadherin and N-Cadherin after treatment for 12-48 h compared to untreated (DMSO treated) HepG2 cells (FIG. 4). The expression of vimentin was not induced by B[a]P (data not shown). These results indicate that AhR activation by B[a]P not only induces the TGF-β1 transcriptional expression, but it is also able to activate TGF-β1 signaling pathway. Moreover, these results show that AhR and TGF-β1 signaling pathway crosstalk to regulate the expression of L1.

B[a]P-regulated L1 expression is via TGF-β1 signaling: Having identified that AhR activation by B[a]P induces the activation TGF-β1 signaling pathway, next objective was to determine what role this signaling pathway play in B[a]P-induced L1 expression. Toward this end, whether important downstream targets of TGF-β1 signaling pathway such as TGFBR1, Smad2 and Smad3 are required for B[a]P-induced L1 mRNA expression was investigated. Expression of each of these molecules was knocked down by transfection of the corresponding siRNAs in HepG2 cells, and immunoblot analysis confirm >80% reduction in the expression levels compared with controls (FIG. 5B). Inhibition of TGFBR1 expression caused a partial decrease in B[a]P-induced L1 mRNA expression (FIG. 5A). Inhibition of Smad2 and Smad3 caused a completely block in (p<0.05) in B[a]P-induced L1 mRNA expression, as well as a slight reduction in baseline L1 mRNA expression (FIG. 5A). All these results indicate that B[a]P-induced L1 expression requires TGF-b1 signaling pathway.

Expression of L1 proteins correlate with TGF-β1 signaling pathway hepatoma-cellular: The above results indicate that AhR activation by B[a]P leads to L1 expression via TGF-β1 signaling pathways acting through Smad-mediated. To further support these results, whether the expression of L1 proteins (ORF-1 and ORF-2) overlap with the activation TGF-β1 signaling pathway was analyzed in human tumor liver tissue from different stages. Extracts of total proteins from human liver hepatocellular carcinoma stages I, II, III, IV, and normal tissue were analyzed by immunoblotting (FIG. 6). Immunoblotting analysis showed that normal liver presented a high expression of E-Cadherin, but the expression of ORF1p and ORF2p proteins was not detected (FIG. 6A). ORF-1 was detected in liver tumor early-stages I, II and IV, and ORF-2 was observed in stage I and IV (FIG. 6A). All the human hepatocellular carcinoma stages presented EMT phenotype-low expression (or not expression) of E-cadherin and a high expression of vimentin (FIG. 6A). A high increase in the phosphorylation levels of Smad2 was observed in stages I, II and IV compared with the normal and the tumor stages III (FIG. 6B), and overlapping with the expression of ORF-1 (FIG. 6A). The results show that L1 expression can be regulated via TGF-β1 signaling pathways acting through Smad-mediated in liver cancer.

L1 levels in COPD patients: Data for L1 levels in COPD patients are summarized in the following four tables.

TABLE A
L1 by Sex, Smoking Status, BMI

| | Mean (SD) | Min | Max | P-value* |
|---|---|---|---|---|
| All (N = 428) | 46.3 (25.3) | 0 | 144.2 | |
| Sex | | | | 0.010 |
| Females (N = 284) | 44.1 (23.9) | 0 | 137.9 | |
| Males (N = 144) | 50.8 (27.9) | 5.6 | 144.2 | |
| Smoking status | | | | 0.112 |
| Never (N = 178) | 43.8 (23.5) | 0 | 127.4 | |
| Ex (N = 127) | 50.0 (26.8) | 6.3 | 144.2 | |
| Current (N = 123) | 46.2 (26.6) | 8.1 | 137.9 | |
| BMI Category | | | | 0.884 |
| Normal weight (N = 215) | 46.6 (26.2) | 9.5 | 142.9 | |
| Underweight (N = 12) | 38.8 (22.4) | 14.1 | 97.3 | |
| Overweight (N = 157) | 46.1 (23.7) | 0 | 144.2 | |
| Obese (N = 35) | 47.4 (28.2) | 8.1 | 106 | |
| Missing (N = 9) | 48.0 (34.1) | 20.9 | 127.4 | |

TABLE B
Correlation with L1

| | | % | % | | |
|---|---|---|---|---|---|
| | | Predicted | Predicted | FEV1/ | |
| | Age | FEV1 | FVC | FVC | Pack-years |
| Correlation | 0.01 | −0.07 | −0.04 | −0.10 | 0.10 |
| Coefficient | | | | | |
| P-value | 0.838 | 0.151 | 0.450 | 0.042 | 0.045 |
| Correlation | 0.05 | −0.19 | −0.15 | −0.18 | 0.10 |
| Coefficient | | | | | |
| P-value | 0.518 | 0.021 | 0.074 | 0.028 | 0.252 |
| Correlation | −0.01 | 0.03 | 0.04 | −0.02 | 0.01 |
| Coefficient | | | | | |
| P-value | 0.889 | 0.632 | 0.486 | 0.682 | 0.825 |

TABLE C
COPD (FEV1/FVC < 70%) With and without plate adjustment

| | L1 continuous | | | L1 quartiles | | |
|---|---|---|---|---|---|---|
| | OR | 95% CI | p | OR | 95% CI | p |
| Males | | | | | | |
| (N = 143)** | | | | | | |
| Without | 1.02 | 1.00, 1.04 | 0.016 | 5.05 | 0.82, 31.19 | 0.081 |
| adjustment | | | | 3.43 | 0.52, 22.66 | 0.200 |
| | | | | 7.69 | 1.29, 45.88 | 0.025 |
| With | 1.02 | 1.00, 1.04 | 0.016 | — | — | — |
| adjustment | | | | | | |
| Female | | | | | | |
| (N = 284)** | | | | | | |
| Without | 1.00 | 0.99, 1.02 | 0.919 | 0.66 | 0.22, 2.00 | 0.461 |
| adjustment | | | | 1.44 | 0.56, 3.69 | 0.451 |
| | | | | 1.00 | 0.36, 2.82 | 0.996 |
| With | 1.00 | 0.98, 1.02 | 0.946 | 0.49 | 0.14, 1.63 | 0.243 |
| adjustment | | | | 0.96 | 0.33, 2.83 | 0.942 |
| | | | | 0.91 | 0.26, 3.15 | 0.884 |

**Adjusted for age, smoking status, pack-years, BMI category

TABLE D
COPD (FEV1/FVC < LLN) With and without plate adjustment

| | L1 continuous | | | L1 quartiles | | |
|---|---|---|---|---|---|---|
| | OR | 95% CI | p | OR | 95% CI | p |
| Males | | | | | | |
| (N = 143)** | | | | | | |
| Without | 1.03 | 1.01, 1.05 | 0.002 | 5.82 | 0.59, 57.13 | 0.131 |
| adjustment | | | | 3.65 | 0.32, 41.29 | 0.295 |
| | | | | 11.37 | 1.24, 103.99 | 0.031 |
| With | 1.03 | 1.01, 1.05 | 0.011 | 5.65 | 0.54, 58.94 | 0.148 |
| adjustment | | | | 3.19 | 0.26, 39.33 | 0.365 |
| | | | | 8.73 | 0.85, 90.01 | 0.069 |
| Females | | | | | | |
| (N = 284)** | | | | | | |
| Without | 1.01 | 0.99, 1.02 | 0.343 | 0.63 | 0.20, 1.96 | 0.421 |
| adjustment | | | | 1.04 | 0.38, 2.83 | 0.942 |
| | | | | 1.61 | 0.60, 4.35 | 0.344 |
| With | 1.00 | 0.99, 1.02 | 0.588 | 0.50 | 0.14, 1.71 | 0.269 |
| adjustment | | | | 0.78 | 0.26, 2.32 | 0.653 |
| | | | | 1.32 | 0.41, 4.19 | 0.641 |

**Adjusted for age, smoking status, pack-years, BMI category

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

What is claimed is:

1. A method of treating a subject suffering from a clinical condition associated with increased expression of long interspersed nuclear element-1 (“LINE-1”), said method comprising:
determining whether a subject has an increased expression level of long interspersed nuclear element-1 (“LINE-1”); and
treating the subject having an increased expression level of LINE-1 with a
transforming growth factor-β1 (“TGF-β1”) inhibitor,
wherein said clinical condition associated with increased LINE-1 expression is selected from the group consisting of tumors, chronic obstructive pulmonary disease, exposure to environment/toxin, and fibrotic and inflammatory disease.

2. The method of claim 1, wherein said step of determining LINE-1 expression level comprises determining the level of ORF-1 protein (“ORF1p”), determining the level of LINE-1 mRNA, determining the amount of LINE-1 in a cell sample of the subject, determining the level of ORF-2 protein, or a combination thereof.

3. The method of claim 2, wherein said step of determining LINE-1 expression level comprises determining the level of ORF1p.

4. The method of claim 3, wherein said step of determining the level of ORF1p comprises contacting a fluid sample, biopsy sample or other cell or tissue sample of the subject with an ORF1p antibody.

5. The method of claim 1, wherein said clinical condition comprises cancer or other chronic disease as defined earlier.

6. The method of claim 5, wherein cancer is selected from the group consisting of lung cancer, bladder cancer, esophageal cancer, gastric cancer, hepatic cancer, breast cancer, prostate cancer, colorectal cancer, testicular cancer, renal cancer, chronic lymphocytic leukemia, chronic myeloid leukemia, ovarian carcinomas, and human papilloma virus related cancers.

7. The method of claim 1, wherein said step of determining whether the subject has an increased level of LINE-1 expression comprises comparing the LINE-1 expression level in the subject with a control LINE-1 expression level.

8. The method of claim 1, said fibrotic and inflammatory disease comprises inflammatory disease of the digestive tract, the genitourinary tract, the lung, the kidney or the liver.

9. The method of claim 1, wherein said fibrotic and inflammatory disease comprises Barrett's Esophagus, colitis, asbestosis, asthma, bronchopulmonary dysplasia, emphysema, bronchitis (acute and chronic), primary ciliary dyskinesia, pulmonary fibrosis, silicosis, or sarcoidosis.

10-14. (canceled)

15. The method of claim 1, wherein the control L1 expression level is a positive control L1 expression level.

16. The method of claim 1, wherein the control L1 expression level is a negative control L1 expression level.

17. The method of claim 1, wherein the control L1 expression level comprises L1 expression level of the cancer patient prior to being diagnosed with the cancer.


SEQUENCE LISTING (16 sequences)

SEQ ID NO: 1 - DNA, 21 bp, Artificial Sequence
  ORF1-Forward Primer Sequence
ccaagttggaaaacactctgc

SEQ ID NO: 2 - DNA, 21 bp, Artificial Sequence
  ORF1-Reverse Primer Sequence
tgtggcgttctctgtatttcc

SEQ ID NO: 3 - DNA, 22 bp, Artificial Sequence
  ORF2-Forward Primer Sequence
tcgacacatacactctcccaag

SEQ ID NO: 4 - DNA, 20 bp, Artificial Sequence
  ORF2-Reverse Primer Sequence
tggtcctggactctttttgg

SEQ ID NO: 5 - DNA, 25 bp, Artificial Sequence
  TGFb1-Forward Primer Sequence
ggataccaactattgcttcagctcc

SEQ ID NO: 6 - DNA, 25 bp, Artificial Sequence
  TGFb1-Reverse Primer Sequence
aggctccaaatataggggcagggtc

SEQ ID NO: 7 - DNA, 20 bp, Artificial Sequence
  GAPDH-Forward Primer Sequence
gatcatcagcaatgcctcct

SEQ ID NO: 8 - DNA, 20 bp, Artificial Sequence
  GAPDH-Reverse Primer Sequence
tgtggtcatgagtccttcca

SEQ ID NO: 9 - DNA, 21 bp, Artificial Sequence
  AHR-siRNA Sense Sequence
cggaugaaauccugacguatt

SEQ ID NO: 10 - DNA, 21 bp, Artificial Sequence
  AHR-siRNA Antisense Sequence
uacgucaggauuucaucagtt

SEQ ID NO: 11 - DNA, 21 bp, Artificial Sequence
  TGFBR1-siRNA Sense Sequence
caguuacuguggaaggaautt

SEQ ID NO: 12 - DNA, 21 bp, Artificial Sequence
  TGFBR1-siRNA Antisense Sequence
auuccuuccacaguaacugtg

SEQ ID NO: 13 - DNA, 21 bp, Artificial Sequence
  SMAD2-siRNA Sense Sequence
gcuucucugaacaaaccagtt

SEQ ID NO: 14 - DNA, 21 bp, Artificial Sequence
  SMAD2-siRNA Antisense Sequence
cugguuuguucagagaagctg

SEQ ID NO: 15 - DNA, 21 bp, Artificial Sequence
  SMAD-siRNA Sense Sequence
ggcccagugcauaugcaautt

SEQ ID NO: 16 - DNA, 21 bp, Artificial Sequence
  SMAD3-siRNA Antisense Sequence
auugcauaugcacugggcctc